(19) United States
(12) Patent Application Publication
(10) Pub. No.: US 2014/0370131 A1
(43) Pub. Date: Dec. 18, 2014

(54) Pharmaceutical Composition for Protecting Brain Neurons Comprising Plumula Nelumbinis Extract as Active Ingredient

(71) Applicant: NATIONAL INSTITUTE OF BIOLOGICAL RESOURCES, Incheon (KR)

(72) Inventors: Eun Sil Kim, Incheon (KR); Choong Je Ma, Gangwon-do (KR); Kyounghee Oh, Seoul (KR)

(73) Assignee: NATIONAL INSTITUTE BIOLOGICAL RESOURCES, Incheon (KR)

(21) Appl. No.: 14/378,972

(22) PCT Filed: Jan. 18, 2013
(86) PCT No.: PCT/KR2013/000396

§ 371 Date: Aug. 15, 2014

(30) Foreign Application Priority Data
Feb. 27, 2012 (KR) 101012-0019799

(51) Int. Cl. A61K 36/48; A23L 1/30

(52) U.S. Cl. CPC A61K 36/48; A23L 1/3002; A23V 2002/00
USPC 424/757

(57) ABSTRACT

The present invention relates to a pharmaceutical composition and food composition comprising Nelumbo nucifera seed extract as an active ingredient for the protection of brain nerve cells. Particularly, the composition is characterized by having an activity of removing reactive oxygen species (ROS) or an antioxidant activity. More particularly, the antioxidant activity is characterized by the activity of removing 2,2-diphenyl-1-picrylhydrazyl (DPPH) radicals or the activity of removing hydrogen peroxide (H2O2). Also, the present invention is extracted from a natural substance and is thus safe for the human body and provides fundamental data to the food and pharmaceutical fields, which use products derived from natural substances.

### CROSS REFERENCE TO RELATED APPLICATIONS AND CLAIM OF PRIORITY

This patent application claims benefit under 35 U.S.C. 119(e), 120, 121, or 365(c), and is a National Stage entry from International Application No. PCT/KR2013/000396, filed 18 Jan. 2013, which claims priority to Korean Patent Application No. 10-2012-0019799, filed 27 Feb. 2012, entire contents of which are incorporated herein by reference.

### BACKGROUND

1. Field of the Invention

The present invention relates to a pharmaceutical composition for brain neurons, which includes a Plumula nelumbinis extract as an active ingredient.

2. Description of the Prior Art

All parts of a lotus (Nelumbo nucifera) belonging to the family Nelumbonaceae are known to have very excellent effects in Chinese medicine. As is known in the art, roemerine and nuciferine, found in a lotus, have very excellent analgesic and sedative effects, etc., and are used in folk remedies for pneumonia, bronchial asthma, gonorrhea, tonic, indigestion, and snake or insect bites. In addition, they aid in energy boosting, fatigue recovery, and tranquilization, and thus long-term intake thereof promotes health. As used herein, the term “Nelumbinis semen” refers to the peeled and dried seed of a lotus, and the term “Plumula nelumbinis” refers to the dried plumule and radical in the mature seed of a lotus. Nelumbinis semen has little or no odor, is sweet in taste, and thus results in less rejection when it is included in Chinese medicines or foods. Nelumbinis semen is oval or ball-shaped and has a small round protrusion at one end. The outer surface thereof is yellowish brown or reddish brown in color and has light gray powder, a fine vertical pattern and a relatively distinct vein-like pattern. The shell of the lotus seed is thin, is yellowish brown in color and is not easily peeled off. The lotus seed includes two yellowish-white thick cotyledons, and there is a green embryo (Plumula nelumbinis) in the middle of the lotus seed.

Recently, increases in average life expectancy, resulting from increases in the standard of living, have promulgated a higher ratio of the elder population. With respect to the causes of death for Koreans, brain diseases, such as stroke, dementia, mental disorder, and behavior disorder, are the leading causes of death following cancer and cardiovascular diseases, and rank among the highest in deaths caused by single-organ diseases (Korea Statistical Yearbook, the number of deaths according to sex, age and cause, 1996). Typical brain diseases include Alzheimer's disease, multiple sclerosis, Parkinson's disease, stroke, ischemia, and the like. Among them, senile brain diseases, including Alzheimer's disease, Parkinson's disease, and stroke, are mainly caused by oxidative stress involving radical formation in brain cells (Smith, M.A. J. Neurochem. 1997, Supp. S1, 69, 19).

As used herein, the term “oxidative stress” means that cells or tissues are damaged by toxic free radicals. Neuronal damage caused by oxidative stress is known to be related to brain cell damage, which occurs during the normal aging process, and to neurodegenerative brain diseases, including Alzheimer's disease, Parkinson's disease, Lou Gehrig's disease, dementia, and the like. In particular, it is known that brain tissue is susceptible to the attack of free radicals, because brain neurons lack a sufficient defense mechanism, contain easily oxidizable long chain unsaturated fatty acids at high concentrations, and metal ions (e.g., iron (Fe²⁺) and copper (Cu²⁺)) acting as catalysts for radical formation.

It was reported that neurodegenerative brain diseases are mainly attributable to oxidative stress caused by the accumulation of reactive oxygen species (ROS) (Olney, J. W. et al. Brain Res. 1974, 77, 507-512). In addition, glutamate, an amino acid, is a major excitatory neurotransmitter, and an excessive concentration of glutamate in the central nervous system inhibits the absorption of N-acetyl cysteine thereby causing oxidative stress. Until now, various natural antioxidants have been reported in terms of antioxidative protection against oxygen radicals that are produced in cells. However, these antioxidants are related to the antioxidant functions of Chinese herbal materials or food additives and have mostly been studied in liver tissue (Park, J. C. et al. J. Korean Soc. Food Nutr. 1996, 25, 588-592; Kim, M. R. et al. J. Food. Sci. Nutr. 1997, 2, 207; Kim, Mee Ree, et al. Food Res. Intl. 1999, 31(5), 389-394). Further, studies conducted to protect the brain using bio-resources are insufficient.

Korean Patent Application No. 10-2009-0077620 discloses a pharmaceutical composition for the prevention and treatment of stress and panic disorders, which include Nelumbinis semen. Korean Patent Registration No. 10-0751047 discloses a food composition for preventing and relieving hangovers, which includes Nelumbinis radix or Plumula nelumbinis, and Korean Patent Registration No. 10-0949926 discloses a functional cosmetic composition having anti-inflammatory, antioxidant, whitening, and antibacterial effects, which includes a combination of Chinese herbal extracts including a Nelumbinis semen extract. However, these patent documents do not disclose that a Plumula nelumbinis extract has activity for protecting brain neurons.

### SUMMARY OF THE INVENTION

In one embodiment, provided is a pharmaceutical composition for the protection of brain neurons, which includes a Plumula nelumbinis extract as an active ingredient.

In another embodiment, provided is a food composition for the protection of brain neurons, which includes a Plumula nelumbinis extract as an active ingredient.

In still yet another embodiment, provided is a method for producing a composition for the protection of brain neurons, the method including adding an organic solvent to Plumula nelumbinis to extract a fraction of the Plumula nelumbinis.

Other aspects provided herein will become more apparent from the following drawings, detailed description, and the appended claims.

### BRIEF DESCRIPTION OF THE DRAWINGS

FIG. 1 is a graph showing the results of measuring the brain neuron-protecting activity of each part of a lotus flower.

FIG. 2 is a graph showing the results of measuring the brain neuron-protecting activity of each of the Plumula nelumbinis fraction layers.

FIGS. 3A to 3D show the state of HT22 cells in an experiment measuring brain neuron-protecting activity. (FIG. 3A: control group, FIG. 3B: cells treated with glutamate alone; FIG. 3C: positive control group, and FIG. 3D: cells treated with an ethyl acetate (EtOAc) extract of Plumula nelumbinis.)

FIGS. 4A and 4B show the results of culturing the HT22 cell line and the morphology of the cells.

FIG. 5 shows the results of measuring the reactive oxygen species (ROS) scavenging activity of an ethyl acetate (EtOAc) extract of Plumula nelumbinis.

FIG. 6 shows the results of measuring the DPPH radical scavenging activity of an ethyl acetate (EtOAc) extract of Plumula nelumbinis.

FIG. 7 shows the results of measuring the hydrogen peroxide (H₂O₂) scavenging activity of an ethyl acetate (EtOAc) extract of Plumula nelumbinis.

FIGS. 8A and 8B show oral administration to male ICR mice.

FIGS. 9A-9C show performing a Moths water maze test.

FIGS. 10A through 10H shows graphs of the results of measuring the average swimming time in a memory acquisition test. (FIG. 10A: control; FIG. 10B: seopolamine treatment (negative control): FIG. 10C: Donepezil treatment (positive control); FIG. 10D: 3 mg/kg of the Plumula nelumbinis extract; FIG. 10E: 10 mg/kg of the Plumula nelumbinis extract; FIG. 10F: 30 mg/kg of the Plumula nelumbinis extract; FIG. 10G: 100 mg/kg of the Plumula nelumbinis extract and FIG. 10H: 200 mg/kg of the Plumula nelumbinis extract.

FIGS. 11A through 11D shows the results of measuring the average swimming distance in the memory acquisition test.

### DETAILED DESCRIPTION

In a first aspect, the present invention provides a pharmaceutical composition for the protection of brain neurons, which includes a Plumula nelumbinis extract as an active ingredient. Specifically, the composition has reactive oxygen species (ROS) scavenging activity or antioxidant activity. More specifically, the antioxidant activity is 2,2-diphenyl-1-picrylhydrazyl (DPPH) radical scavenging activity or hydrogen peroxide (H₂O₂) scavenging activity.

As used herein, the term “antioxidant” refers to an action or effect of inhibiting oxidation.

In accordance with an embodiment of the present invention, the composition of the present invention has reactive oxygen species (ROS) scavenging activity, 2,2-diphenyl-1-picrylhydrazyl (DPPH) radical scavenging activity, or hydrogen peroxide (H₂O₂) scavenging activity.

As used herein, the term “oxidation” refers to a chemical reaction in which any atom, molecule or ion loses an electron or any material bonds with oxygen to remove hydrogen. When oxidation occurs, a free radical occurs to induce a chain reaction that damages cells. As used herein, the term “antioxidant” refers to an effect or activity of removing a free radical to stop a chain reaction to thereby suppress an oxidation reaction.

The term “extract” as used herein when referring to the Plumula nelumbinis extract is meant to include a material resulting from the treatment of Plumula nelumbinis with an extraction solvent.

As used herein, the term “pharmaceutically effective amount” refers to an amount sufficient to achieve the antioxidant effect or activity of the Plumula nelumbinis extract.

In one embodiment, the content of the Plumula nelumbinis extract in the pharmaceutical composition of the present invention may be 0.001-99.999 wt %, and 0.1-90 wt %. However, the content of the extract is not limited to the above-described ranges, and may vary depending on the condition of the patient and the type and severity of disease.

In another embodiment, when the composition of the present invention is prepared as a pharmaceutical composition, the pharmaceutical composition includes a pharmaceutically acceptable carrier. Examples of pharmaceutically acceptable carriers used in pharmaceutical compositions of the present invention include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxylbenzoate, talc, magnesium stearate, and mineral oil, all generally used in formulations. The pharmaceutical composition of the present invention may further include a lubricant, a wetting agent, a sweetening agent, a flavoring agent, an emulsifying agent, a suspending agent, a preservative, or the like. Suitable pharmaceutically acceptable carriers and formulations are described in detail in Remington's Pharmaceutical Sciences (19th ed., 1995).

In yet another embodiment, the pharmaceutical composition of the present disclosure may be administered orally or parenterally.

In one embodiment, the appropriate dosage of the pharmaceutical composition of the present disclosure may vary depending on various factors including, but without limitation, the method of formulation, the mode of administration, the patient's age, body, weight, and sex, pathological condition, diet, the time of administration, the route of administration, excretion rate, and response sensitivity. In one embodiment, the general dosage of the pharmaceutical composition of the present invention me be 0.001-1000 mg/kg for adults.

In another embodiment, the pharmaceutical composition of the present invention may be formulated as a unit dosage form or a multiple dosage form using a pharmaceutically acceptable carrier and/or excipient according to a method that can be easily carried out by those skilled in the art. In one embodiment, the formulation may be in the form of a solution, an oily or aqueous medium, suspension, syrup, emulsion, extract, dust, powder, granule, tablet or capsule, and may further include a dispersant or a stabilizer.

In a second aspect, the present invention provides a food composition for the protection of brain neurons, which includes a Plumula nelumbinis extract as an active ingredient. Specifically, the composition has reactive oxygen species (ROS) scavenging activity or antioxidant activity. More specifically, the antioxidant activity is DPPH (2,2-diphenyl-1-picrylhydrazyl) radical scavenging activity or hydrogen peroxide (H₂O₂) scavenging activity.

In one embodiment, the composition of the present invention is prepared as a food composition, and may include, in addition to the Plumula nelumbinis extract, components that are commonly added in the preparation of foods. Examples of such components may include, without limitation, proteins, carbohydrates, fats, nutrients, seasonings, and flavoring agents. Examples of the carbohydrate may include, without limitation, sugars such as monosaccharides, for example, glucose, fructose, etc., disaccharides, for example, maltose, sucrose, oligosaccharide, etc., or polysaccharides, for example, dextrin, cyclodextrin, etc., and sugar alcohols such as xylitol, sorbitol, erythritol, etc. The flavoring agent may be a natural flavoring agent [thaumatin, stevia extract (e.g., rebaudioside A, glycynhizin, etc.)] or a synthetic flavoring agent (e.g., saccharin, aspartame, etc.). For example, in one embodiment, when the food composition of the present invention is prepared as a drink, it may further include, in addition to the Plumula nelumbinis extract, citric acid, liquid fructose, sugar, glucose, acetic acid, malic acid, fruit juice, eucommia extract, jujube extract, licorice extract, or the like.

Furthermore, in another embodiment, the composition of the present invention may contain various nutrients, vitamins, minerals (e.g., electrolytes), synthetic and natural flavoring agents, colorants, fillers (e.g., cheese, chocolate, etc.), pectic acid and its salts, alginic acid and its salts, organic acids, protective colloidal thickeners, pH modifiers, stabilizers, preservatives, glycerin, alcohol, and carbonating agents for carbonated beverages. Additionally, in yet another embodiment, the composition of the present invention may contain fruit flesh that is used for the preparation of natural fruit juice, fruit juice beverages, or vegetable-based beverages. These additives may be used alone or in combination. In yet still another embodiment, the content of these additives within the composition of the present invention is not critical, but generally are in the range of 0 to 20 parts by weight based on 100 parts by weight of the composition.

In a third aspect, the present invention provides a method for preparing a composition for the protection of brain neurons, the method includes adding an organic solvent to Plumula nelumbinis to extract a fraction of the Plumula nelumbinis.

In one embodiment, the inventive method for preparing a composition for the protection of brain neurons includes the steps of: (a) preparing Plumula nelumbinis; and (b) adding an organic solvent to the Plumula nelumbinis to obtain an organic solvent extract.

In one embodiment, the organic solvent used in the present invention may be selected from various extraction solvents. The organic solvent may be a polar solvent or a non-polar solvent. Suitable examples of polar solvents that may be used in the present invention include, without limitation, water, alcohol (i.e., methanol, ethanol, propanol, butanol, n-propanol, iso-propanol, n-butanol, 1-pentanol, 2-butoxyethanol, or ethylene glycol), acetic acid, dimethylformamide (DMF) and dimethylsulfoxide (DMSO). Suitable examples of non-polar solvents that may be used in the present invention include, without limitation, acetone, acetonitrile, ethyl acetate, methyl acetate, fluoroalkane, pentane, hexane, 2,2,4-trimethylpentane, decane, cyclohexane, cyclopentane, diisobutylene, 1-pentene, 1-chlorobutane, 1-chloropentane, o-xylene, diisopropyl ether, 2-chloropropane, toluene, 1-chloropropane, chlorobenzene, benzene, diethyl ether, diethyl sulfide, chloroform, dichloromethane, 1,2-dichloroethane, aniline, diethylamine, ether, carbon tetrachloride, and tetrahydrofuran (THF).

In another embodiment, the organic extraction solvent used in the present invention may be selected from the group consisting of C1-C4 anhydrous or hydrated lower alcohol (e.g., methanol, ethanol, propanol, butanol, etc.), a mixed solvent including at least one of the lower alcohols and water, acetone, ethyl acetate, chloroform, butyl acetate, 1,3-butylene glycol, hexane, and diethyl ether. Alternatively, water may be used instead of the organic extraction solvent. In yet another embodiment, the organic extraction solvent used in the present invention is ethyl acetate.

As used herein, the term “fraction” is meant to include not only a fraction obtained using an organic extraction solvent, but also a material obtained by purifying the obtained fraction.

In one embodiment, the inventive method for preparing a composition for the protection of brain neurons includes the steps of: (a) preparing Plumula nelumbinis; (b) adding methanol to the Plumula nelumbinis to form a mixture; (c) sonicating the mixture; (d) isolating and purifying a methanol fraction from the sonicated mixture; (e) adding ethyl acetate to the isolated and purified methanol fraction; and (f) isolating and purifying an ethyl acetate fraction from the mixture of step (e).

Hereinafter, the present invention will be described in further detail with reference to the following examples. It is to be understood, however, that these examples are for illustrative purposes only, and are not intended to limit the scope of the present invention

### Example 1

### Extraction of a Fraction Layer of Lotus Flower

Among the parts of a lotus flower, the lotus seed, the seed testa, the embryo (Plumula nelumbinis) and the cotyledon were extracted via sonication.

The extraction solvent used was 80% methanol, and the solvent was added in an amount of 1L per 100 g of each sample.

Each sample was extracted at a sonication frequency of 42 kHz, three times for 90 minutes each.

The yields for the extraction of the lotus flower parts were 4.15% for the lotus seed, 9.25% for the seed testa, 44.06% for the embryo (Plumula nelumbinis), and 19.76% for the cotyledon.

The Plumula nelumbinis was fractionated sequentially using hexane, CHCl₃, EtOAc and n-BuOH solvents in increasing order of polarity. The amounts of the obtained fractions were 0.76 g for the hexane layer, 1.00 g for the CHCl₃ layer, 0.41 g for the EtOAc layer, and 2.65 g for the n-BuOH layer.

### Example 2

### Brain Neuron-Protecting Activity of each Part of Lotus Flower

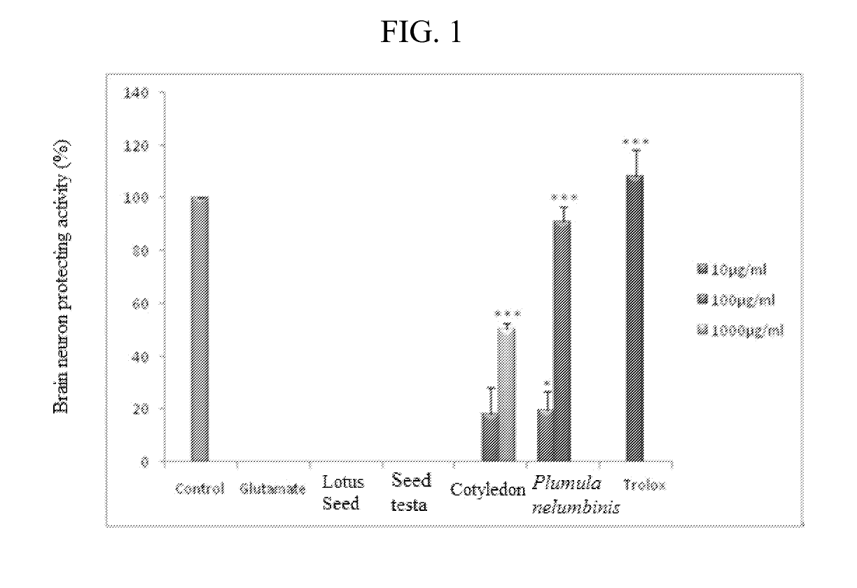

Brain neuron protecting activity was measured in HT22 cells derived from the mouse hippocampus. The survival rate of HT22 cells was measured by an MTT assay. For the MTT assay, HT22 cells were seeded onto a 48-well plate at a density of 3×10⁴ cells/well, and then cultured at 37° C. for 24 hours. After culture, the cells were treated with varying concentrations of each test sample and incubated for 1 hour. Then, the cells were treated with glutamate and incubated at 37° C. for 24 hours. Then, an MTT solution was added to the cells, and after 3 hours, the cells were lysed with DMSO. Next, the absorbance at 570 nm was measured using an enzyme-linked immunosorbent assay (ELISA) reader. As a result, among the extracts of the parts of the lotus flower, the extract of the Plumula nelumbinis showed a brain neuron protecting activity of 90.84% at a concentration of 100 μg/ml (FIG. 1).

### Example 3

### Brain Neuron Protecting Activity of each of Plumula nelumbinis Fraction Layers

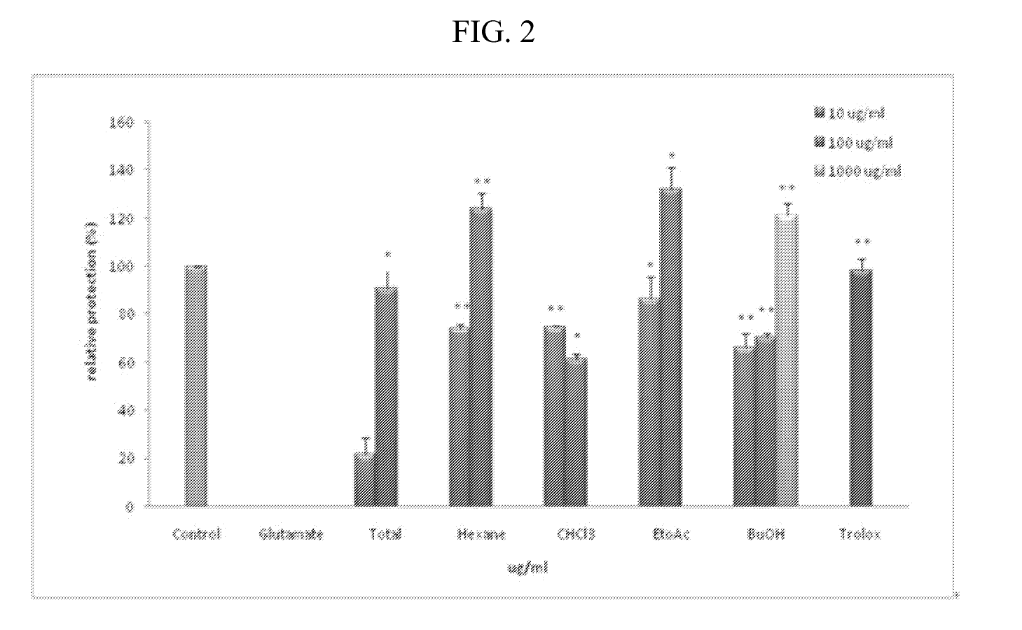

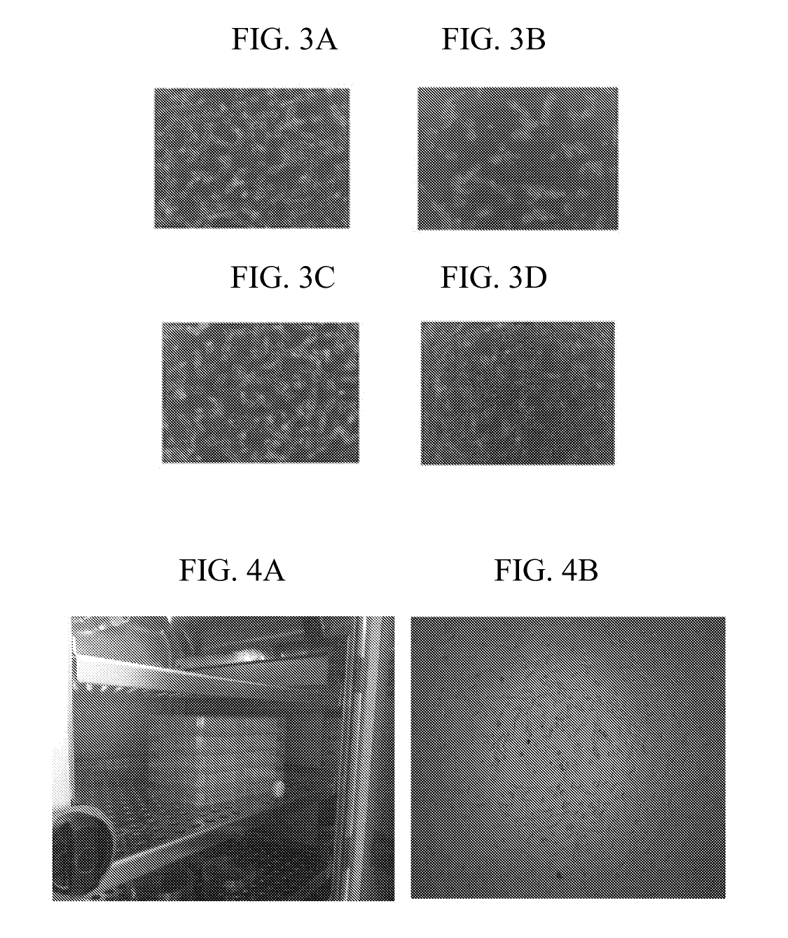

The Plumula nelumbinis extract confirmed to have the highest brain neuron-protecting activity among extracts of the parts of the lotus flower was fractionated, and the cell protecting activity of each of the fraction layers was measured. As a result, the EtOAc layer of the Plumula nelumbinis extract showed the highest cell protecting activity (131.82%) at a concentration of 100 μg/ml (FIGS. 2 and 3).

### Example 4

### HT22 Cell Culture and MTT Assay

To evaluate the anti-dementia activity of a Plumula nelumbinis fraction, an extract obtained by extracting Plumula nelumbinis with 80% methanol was used in the experiment after it was concentrated under reduced pressure and freeze-dried. HT22 cells derived from the mouse hippocampus were cultured in Dulbecco's modified Eagle's medium (DMEM) containing 10% fetal bovine serum and 1% penicillin/streptomycin (P/S) in an incubator at 37° C. while continuously supplying a mixed gas of air (95%) and CO₂ (5%). When the cells grew to a specific confluence, the cells were subcultured using 0.25% trypsin, and the survival rate of the HT22 cells was measured by an MTT assay. For the MIT assay, the HT22 cells were seeded onto a 48-well plate at a density of 3×10⁴ cells/well, and then cultured at 37° C. for 24 hours. After culture, the cells were treated with varying concentrations of the test sample and incubated for 1 hour. Then, the cells were treated with glutamate and incubated at 37° C. for 24 hours. Then, an MIT solution was added to the cells, and after 3 hours, the cells were lysed with DMSO. Next, the absorbance at 570 nm was measured using an enzyme-linked immunosorbent assay (ELISA) reader. As a positive control drug, the tocopherol derivative Trolox was used. In addition, all the experimental values were expressed as the average of cell protection ratios relative to the control, and each experiment was performed in triplicate.

### Example 5

### Measurement of Reactive Oxygen Species (ROS)

Mechanisms of cell death caused by glutamate in HT22 cells include cell death induced by oxidative stress, which generates reactive oxygen species (ROS). In order to investigate the HT22 cell protection-associated mechanism, the reactive oxygen species (ROS) scavenging activity of the EtOAc extract of Plumula nelumbinis was measured by measuring the survival rate of HT22 cells.

HT22 cells were treated with the sample, Trolox and glutamate, and then incubated at 37° C. for 8 hours. After incubation, 10 μM of dichlorofluorescin diacetate (DCF-DA) was added to the cells, which were then incubated for 1 hour. After the reaction with DCF-DA, the medium was removed, and the cells were lysed with PBS containing 1.0% Triton X-100 at 37° C. for 10 minutes. Fluorescence was measured with the excitation wavelength at 490 nm and the emission wavelength at 525 nm.

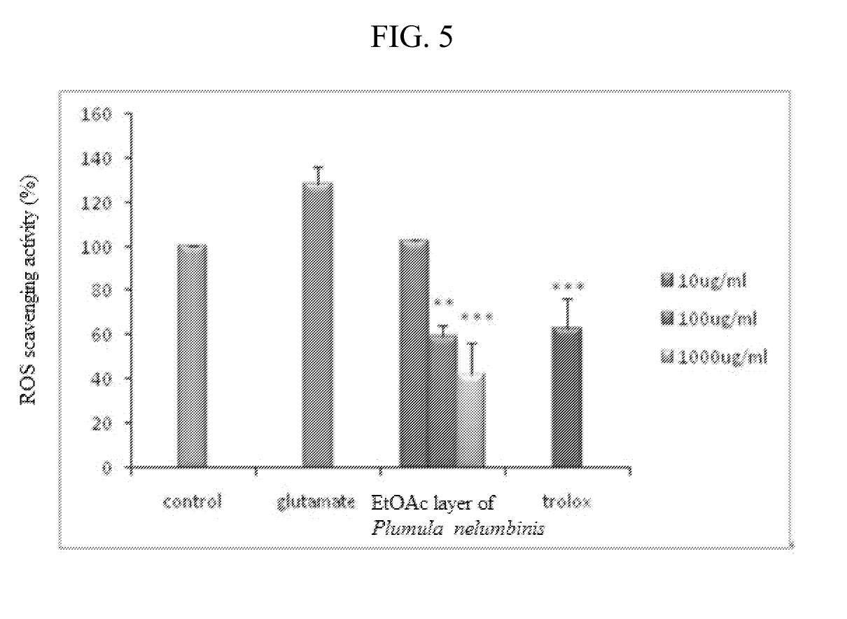

As a result, the EtOAc layer of Plumula nelumbinis showed ROS scavenging activities of 59.19% and 45.55% at 100 ug/ml and 1000 ug/ml, respectively. This suggests that the brain neuron protecting activity of the EtOAc layer of Plumula nelumbinis is associated with ROS scavenging activity (FIG. 5).

### Example 6

### Measurement of 2,2-diphenyl-1-picrylhydrazyl (DPPH) radical scavenging activity

The test sample was weighed, dissolved in methanol or ethanol, and then filtered through a 0.45-μm filter. The prepared sample was serially diluted. Varying concentrations of 150 μl of each diluted sample were added to a 96-well micro plate, followed by addition of DPPH. The 96-well micro plate was placed in a dark room and allowed to stand for 30 minutes. The absorbance at 517 nm was measured. As a result, the EtOAc layer of Plumula nelumbinis showed an IC₅₀ value of 90.89 ug/ml, and vitamin C (ascorbic acid), used as a positive control, showed an IC₅₀ value of 27.73 μg/ml.

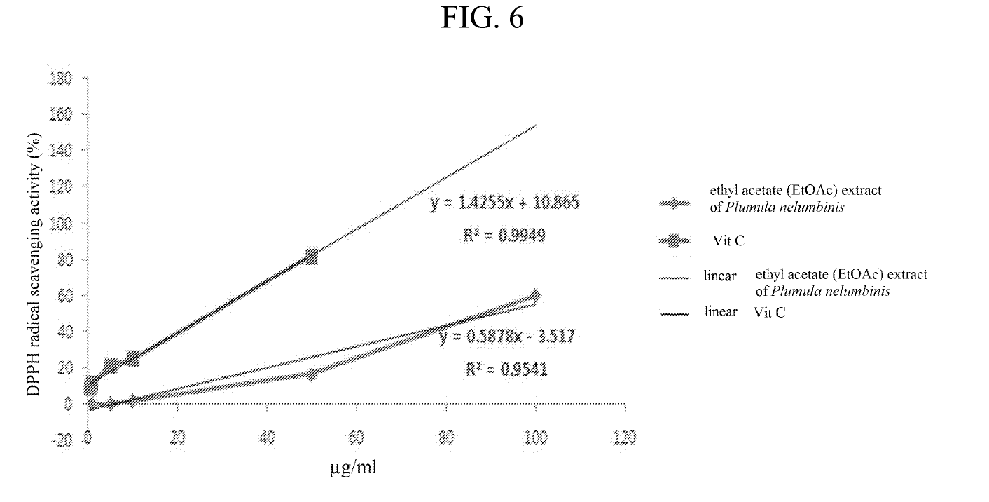

The free radical scavenging activity of the EtOAc layer of Plumula nelumbinis confirmed to have high cell-protecting activity was measured, and as a result, it showed an IC₅₀ of 90 ug/ml (FIG. 6). This free radical scavenging activity is somewhat lower than the brain neuron protecting activity, and this is believed to be because the content of a phenolic compound in the EtOAc layer is low.

### Example 7

### Measurement of Hydrogen Peroxide (H₂O₂) Scavenging Activity

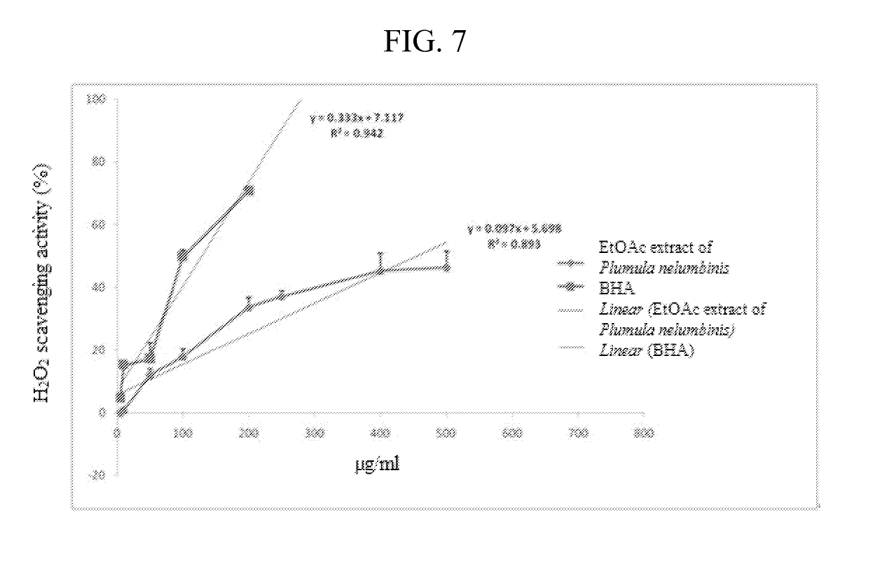

Hydrogen peroxide (H₂O₂) scavenging activity was measured in a 2,2-azinobis(3-ethylbenzthiazolin)-6-sulfonicacid (ABTS)-peroxidase system according to the Muller method. Each sample was diluted at varying concentrations. 80 μL of each sample solution, 20 μL of 10 mM H₂O₂ and 100 μL of phosphate buffer (pH 5.0, 0.1 M) were added to a 96-well plate and reacted at 37° C. for 5 minutes. Then, 30 μL of 1.25 mM ABTS and 30 μL of 1 U/mL peroxidase were added thereto and reacted at 37° C. for 10 minutes, and the absorbance at 405 nm was measured using an enzyme-linked immunosorbent assay (ELISA) reader. As a result, the IC₅₀ value of the ethyl acetate (EtOAc) layer of Plumula nelumbinis was 639.67 ug/ml, and the IC₅₀ value of butylated hydroxy anisole (BHA), used as a positive control, was 160.58 ug/ml (FIG. 7).

Among the extracts of the parts of the lotus flower, the extract of the Plumula nelumbinis showed the highest brain neuron-protecting activity, and among the fraction layers of the extract of the Plumula nelumbinis, the EtOAc layer showed the highest brain neuron-protecting activity. In order to investigate the mechanism of the brain neuron-protecting activity of the EtOAc layer of Plumula nelumbinis, the ROS scavenging activity and antioxidant activities (i.e., DPPH radical and hydrogen peroxide scavenging activities) of the EtOAc layer were measured, and as a result, it is believed that the brain neuron-protecting activity of the EtOAc layer of Plumula nelumbinis in HT22 cells that showed cell death caused by glutamate is caused by the antioxidant activity that prevents oxidative stress.

### Example 8

### Morris Water Maze Test

To observe the cell-protecting effect and cognitive function-improving effect of the Plumula nelumbinis extract in vivo using a Morris water maze test, International Cancer Research (ICR) mice having an average weight of about 30 g were purchased and acclimated to the environment of the laboratory for 1 week before use in the experiment. The animal breeding room was maintained at room temperature (ca. 22±2° C.) with 12 h light (200-300 lux)/12 h dark cycles. The animals had free access to food (i.e., 22.1% or more crude protein, 8.0% or less crude fat, 0.6% or more calcium, and 0.4% or more phosphorus; Samyang Corp., Korea) and water. The animals were divided into a control group administered with the same amount of injectable saline without scopolamine (Sigma Aldrich), and a group administered with scopolamine. The group administered with scopolamine was treated with 3, 10, 30, 100 and 200 mg/kg of a 80% methanol extract of Plumula nelumbinis. As a positive control, donepezil was used, which is a cholinesterase inhibitor-based drug approved by the FDA, and is used for the treatment of mild, moderate, or severe Alzheimer's disease and for the alleviation of vascular dementia (i.e., dementia involving cerebrovascular disease).

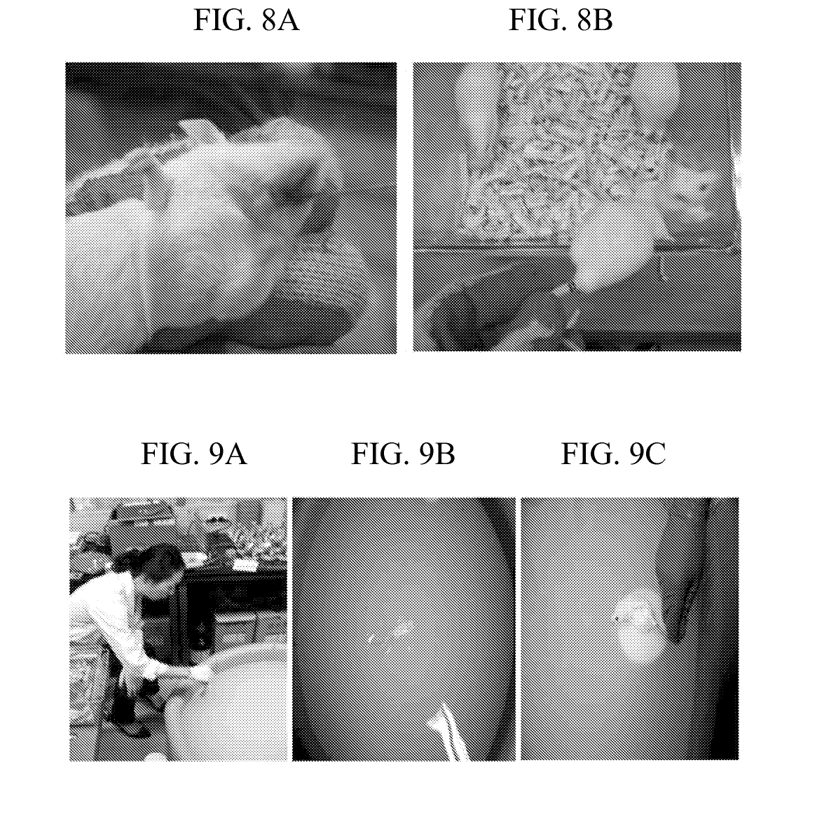

Each of the test samples and the positive control drug was suspended in injectable sterile water and administered at a unit dose of 10 ml/kg. Scopolamine (1 mg/kg) was administered intraperitoneally each day for 4 days, and the test sample and the positive control drug were administered orally (FIGS. 8A and 8B).

In the water maze test, a learning trial was performed on the day before administration of the extract, and then a memory acquisition test was repeated for 4 days. In the learning trial, each test animal was placed into a water pool (200 cm) at one of four release points and allowed to find a platform (200 cm) by swimming for 60 seconds. After finding the platform, the test animal was allowed to rest on the platform for about 10 seconds, and if the test animal failed to find the platform within 60 seconds, it was also allowed to rest on the platform for 10 seconds. After all the test animals were subjected to one learning trial, the learning test was performed in the same manner twice a day for 4 days. The release point was varied each day so as not to overlap.

During the memory acquisition test, 1 mg/kg of scopolamine was administered intraperitoneally once a day for 4 days at 60 minutes after administration of the Plumula nelumbinis extract. 30 minutes after administration of scopolamine, the memory acquisition test was performed. The cognitive ability-improving effect was evaluated by measuring the average swimming time (sec) taken for the test animal to find the platform (sec).

After the learning trial for 1 day, scopolamine was administered to cause damage to cognitive function (memory), and the Moths water maze test was performed to examine the cognitive function-improving effect of the extract. In the memory acquisition test performed over 4 days, the cognitive function-improving effect of the extract was evaluated based on a decrease in each of the average swimming times taken to find the platform and the swimming distance. During the test period, death of the test animal and an abnormal change in the body weight, caused by administration of the Plumula nelumbinis extract, were observed.

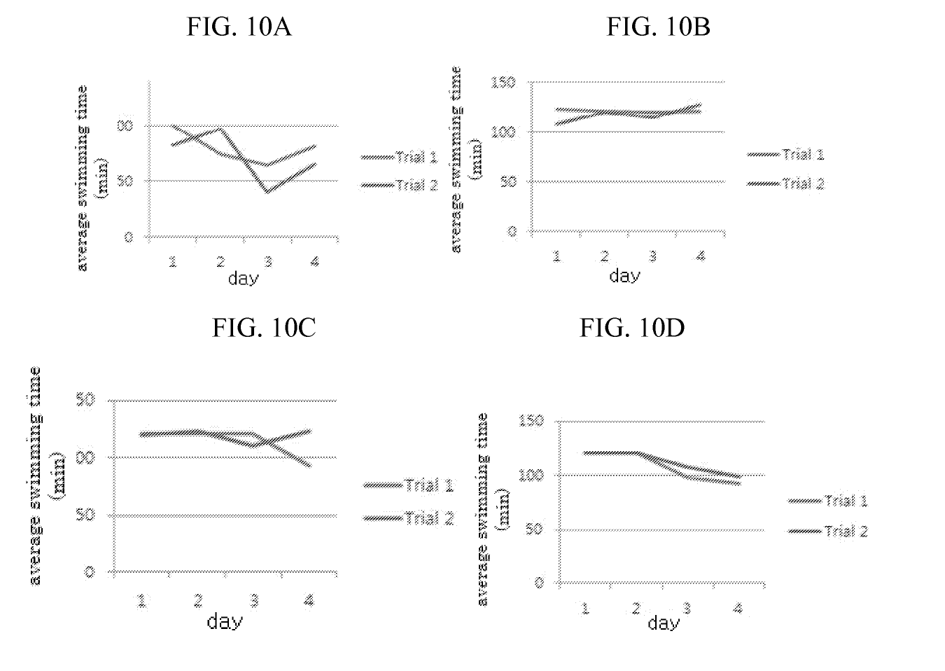

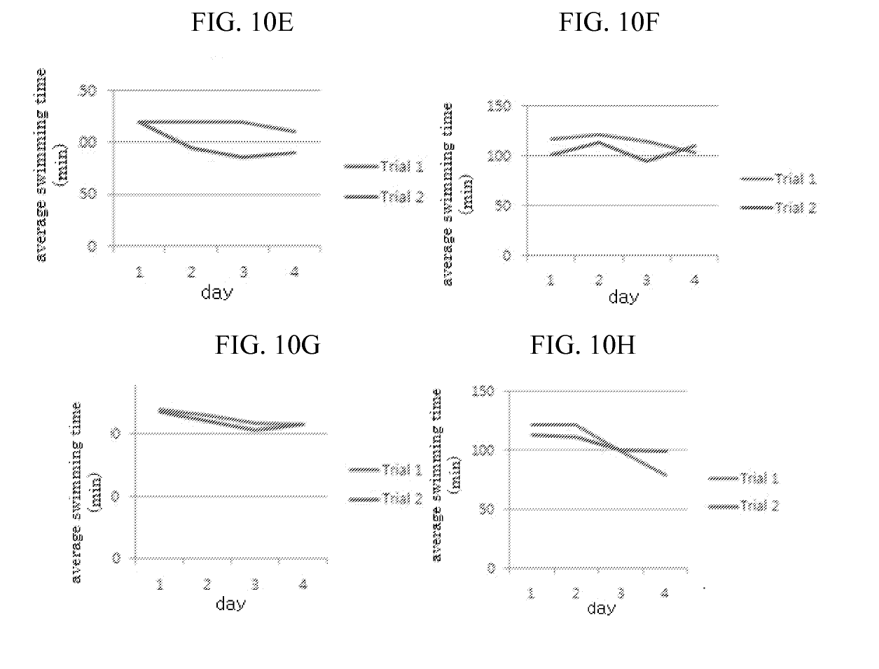

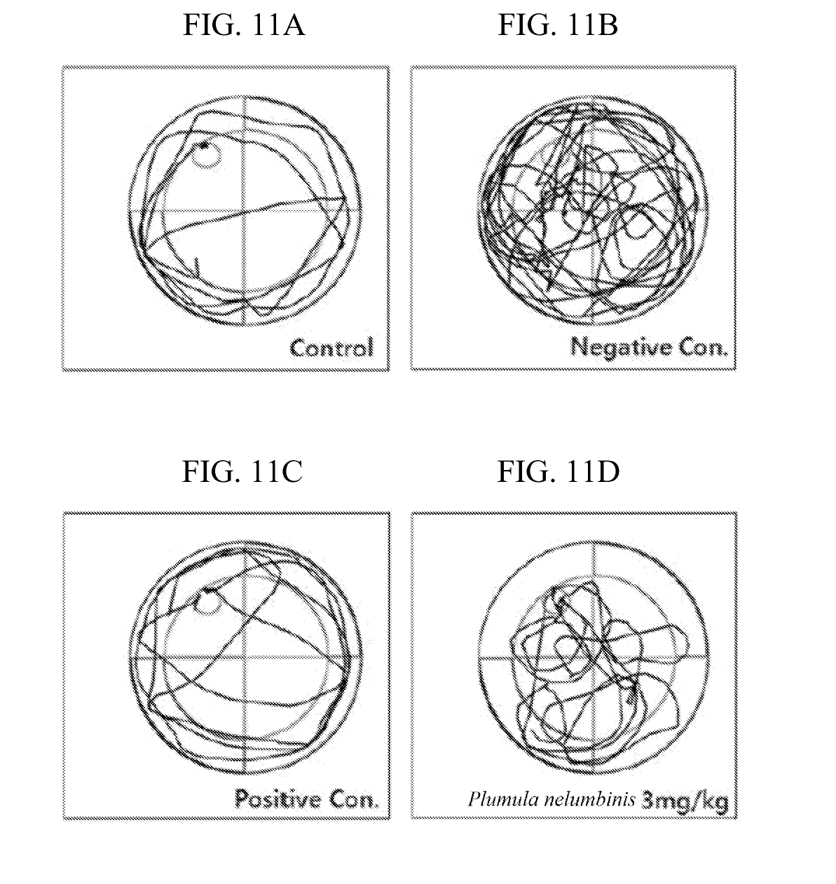

In the results of the memory acquisition test for 4 days, the control group not administered with the test sample and scopolamine, the average swimming time taken for the test animals to find the platform, and the average swimming distance of the test animals were all significantly reduced, whereas in the negative control group administered with scopolamine, the average swimming time and distance increased rather than decreased (FIGS. 10 and 11).

Such results suggest that administration of scopolamine to the mice used as the test animals easily caused damage to cognitive function (memory).

The groups administered with 3, 10, 30, 100, and 200 mg/kg of the extract, and the positive control drug donepezil 90 minutes before administration of scopolamine showed gradual decreases in the average swimming time and the average swimming distance compared to the control group not administered with the test drug. Particularly, other natural materials showing concentration-dependent effects, a low concentration (3 mg/kg) of the Plumula nelumbinis extract showed an effect comparable to high concentrations of the Plumula nelumbinis extract.

Taking the above-described results together, it can be seen that oral administration of the Plumula nelumbinis extract to the mice used as the test animals effectively improved cognitive function (memory) damaged by administration of scopolamine. In addition, it can be seen that the Plumula nelumbinis extract has a significant effect of improving dementia by protecting neuronal cells and shows a significant effect of improving dementia-related diseases in an in vivo test.

In summary:

(i) In one embodiment, the present invention provides a natural extract, Plumula nelumbinis extract, which has excellent brain neuron-protecting activity.

(ii) In another embodiment, the Plumula nelumbinis extract of the present invention has excellent reactive oxygen species (ROS) scavenging activity and antioxidant activity.

(iii) In yet another embodiment, the Plumula nelumbinis extract of the present invention is derived from a natural material, and thus is safe to the human body.

(iv) In still yet another embodiment, the Plumula nelumbinis extract of the present invention has an excellent effect of protecting brain neurons, and thus provides fundamental data in the food and pharmaceutical fields that use natural materials.

1-10. (canceled)

11. A method for preparing a composition, the method comprising:
preparing Plumula nelumbinis; and
adding an organic solvent to the Plumula nelumbinis to obtain an organic solvent extract.

12. The method of claim 11, wherein the organic solvent is any one selected from the group consisting of hexane, chloroform, ethyl acetate, and n-BuOH.

13. The method of claim 11, wherein the organic solvent is ethyl acetate.

14. A method for preparing a composition, the method comprising:
preparing Plumula nelumbinis;
adding methanol to the Plumula nelumbinis to form a mixture;
sonicating the mixture;
isolating and purifying a methanol fraction from the sonicated mixture;
adding ethyl acetate to the isolated and purified methanol fraction; and
isolating and purifying an ethyl acetate fraction from the mixture of step (e).

15. A method of preventing or treating a brain disease, the method comprising:
preparing a composition comprising a Plumula nelumbinis extract as an active ingredient; and
administering to a subject in need thereof the composition.

16. The method of claim 15, wherein the composition is a food composition.

17. The method of claim 15, wherein the preparation comprises:
preparing Plumula nelumbinis; and
adding an organic solvent to the Plumula nelumbinis to obtain the Plumula nelumbinis extract.

18. The method of claim 17, wherein the organic solvent is selected from the group consisting of hexane, chloroform, ethyl acetate, and n-BuOH.

19. The method of claim 17, wherein the organic solvent is ethyl acetate.

20. The method of claim 15, wherein the preparation comprises:
preparing Plumula nelumbinis;
adding methanol to the Plumula nelumbinis to form a mixture;
sonicating the mixture;
isolating and purifying a methanol fraction from the sonicated mixture;
adding ethyl acetate to the isolated and purified methanol fraction; and
isolating and purifying an ethyl acetate fraction from the mixture of step (e) to prepare the Plumula nelumbinis extract.
